(19) United States
(12) Patent Application Publication
(10) Pub. No.: US 2009/0062611 A1
(43) Pub. Date: Mar. 5, 2009

(54) ENDOSCOPE APPARATUS

(75) Inventor: Ryuichi TOYAMA, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., Tokyo (JP)

(21) Appl. No.: 12/186,095

(22) Filed: Aug. 5, 2008

(30) Foreign Application Priority Data
Aug. 29, 2007 (JP) 2007-223258

(51) Int. Cl. A61B 1/015

(52) U.S. Cl. 600/118

(57) ABSTRACT

The present invention is an endoscope apparatus comprising an over tube having a balloon, a fluid suction pipeline provided at the over tube so that a suction opening portion is opened on a proximal end side from the balloon, a suctioning device for suctioning a fluid in the fluid suction pipeline, a pressure/flow-rate sensor for detecting a pressure of the fluid at least at a single position from a lumen to which the over tube is inserted to the suctioning device through the fluid suction pipeline, and a control circuit for controlling a suction pump so that the pressure of the fluid in the lumen in the vicinity of the suction opening portion falls within an appropriate pressure range based on the pressure detected by the pressure/flow-rate sensor.

### CROSS REFERENCE TO RELATED APPLICATION

This application claims benefit of Japanese Application No. 2007-223258 filed in Japan on Aug. 29, 2007, the contents of which are incorporated by this reference.

### BACKGROUND OF THE INVENTION

1. Field of the Invention

The present invention relates to an endoscope apparatus provided with a large diameter portion for gripping a lumen at an intraluminal insertion portion.

2. Description of the Related Art

Endoscopes are used for observing a portion such as the inside of a lumen that can not be visually recognized in a direct way in various fields, such as medicine and industries.

A medical endoscope, for example, is inserted into various lumens of a subject, and an example of use is inserting into a colon, a small intestine and the like. Since such organs have long luminal lengths and complicated shapes with curved portions or bending portions, endoscopes to be used for a large and small intestines have not only a long length of an insertion portion but a devised configuration so that insertion can be made surely.

An example is an over tube disposed on an outer peripheral side of the endoscope insertion portion. That is, the over tube is used when an endoscope is inserted into a lumen such that the endoscope and the over tube are both inserted and then, only the endoscope is further inserted into a lumen while being guided by the over tube or that the over tube and the endoscope are alternately inserted into the lumen further deeply.

At such insertion, in order to ensure the operation, there is a known art that an expandable large-diameter portion (however, the portion becomes the large-diameter portion when expanded) called a balloon is provided at least either one of the endoscope insertion portion, which is an intraluminal insertion portion, and the over tube, which is the intraluminal insertion portion so that a positional relation between the lumen and the intraluminal insertion portion is fixed (gripped) by the balloon. There is also a known usage that after such gripping, by pulling the intraluminal insertion portion toward the hand side, for example, a lumen such as an intestine is pulled toward the hand side and reduced in length and hence, the insertion length of the endoscope insertion portion is shortened.

Japanese Patent Application Laid-Open Publication No. 2007-29556 as a specific example of such an art describes an art of providing a plurality of balloons for holding the shape of a body-cavity lumen at an endoscope-insertion assisting tool serving as an assisting tool used upon insertion of a medical device into a depth of a body-cavity lumen.

Japanese Patent Application Laid-Open Publication No. 2005-205182 describes a so-called double-balloon type endoscope system in which a balloon is provided at a distal end portion of an endoscope and a balloon is also provided at an over tube disposed on an outer peripheral side of the endoscope.

If a lumen is an intestine, for example, its inner diameter is known to be different depending on a patient. Even in a case of a large intestine of the same patient, while a colon, a transverse colon, an ascending colon and the like have larger diameters, a descending colon, a sigmoid colon and the like have smaller diameters, thus the inner diameters are different depending on a portion. However, since an expansion rate of a balloon is limited, it was difficult to effect assured grip for all the intestines having such various inner diameters. Thus, when an intestine having a large inner diameter is to be pulled and reduced in length, the balloon slips with respect to the intestine, which results in a problem that a sufficient reduction operation is difficult and an endoscope can not be inserted properly.

As an example of an art to address such a problem, Japanese Patent Application Laid-Open Publication No. 5-293077 describes an art to further ensure gripping performance, in an intraluminal inserting device having a self-running portion provided with two balloons, by providing a suction unit to at least one of the balloons of the self-running portion that applies a negative pressure to an intraluminal peripheral wall.

However, if suctioning is simply carried out in order to increase a gripping force of the balloon in the art as described in the Japanese Patent Application Laid-Open Publication No. 5-293077, there might be a case where the gripping force is lacking or excessive. For example, if the suctioning force is weak, the gripping force can not be enough, while an excessively strong suctioning force is not preferable, either.

Therefore, an art to have a gripping force in a proper range without lack or excess, when a large-diameter portion such as a balloon is gripped to a lumen using the suctioning art, has been in demand.

### SUMMARY OF THE INVENTION

The present invention has an object to provide an endoscope apparatus that can surely grip a large-diameter portion of an intraluminal insertion portion to a lumen without lack or excess.

In brief the present invention is an endoscope apparatus provided with an intraluminal insertion portion to be inserted into a lumen, a large-diameter portion provided in the intraluminal insertion portion, a fluid suction pipeline provided at least at the intraluminal insertion portion so that a suction opening portion is opened on an outer surface of the intraluminal insertion portion at a proximal end side from the large-diameter portion, a suctioning device to which the proximal end side of the fluid suction pipeline is connected for suctioning fluid in the fluid suction pipeline, a pressure detection portion for detecting a pressure of fluid at least at a single position from the inside of the lumen into which the intraluminal insertion portion is inserted to the suctioning device through the fluid suction pipeline, and a control portion for controlling the suctioning device so that a pressure of the fluid in the lumen close to the suction opening portion becomes an appropriate pressure based on the pressure detected by the pressure detection portion.

The above and other objects, features and advantages of the invention will become more clearly understood from the following description referring to the accompanying drawings.

### BRIEF DESCRIPTION OF THE DRAWINGS

FIG. 1 is a diagram illustrating configuration of an endoscope apparatus in a first embodiment of the present invention;

FIG. 2 is a diagram illustrating configuration of a scope and an over tube in the first embodiment in more detail;

FIG. 3 is a 3-3 sectional view of FIG. 2 illustrating the configuration of the over tube in the first embodiment;

FIG. 4 is a 4-4 sectional view of FIG. 2 illustrating the configuration of the over tube in the first embodiment;

FIG. 5 is a 5-5 sectional view of FIG. 2 illustrating the configuration of the over tube in the first embodiment;

FIG. 6 is a diagram illustrating a state where the scope and the over tube are inserted into a lumen in the first embodiment;

FIG. 7 is a diagram illustrating a state where the scope and the over tube are inserted into the lumen for suctioning in the first embodiment;

FIG. 8 is a diagram illustrating a state where the scope and the over tube inserted into the lumen in a suctioning state are pulled so as to make the lumen straight in the first embodiment;

FIG. 9 is a diagram illustrating a state where the scope is relatively advanced and inserted with respect to the over tube after the lumen is made straight in the first embodiment;

FIG. 10 is a line diagram illustrating an example of control so that the pressure of the suctioning becomes an appropriate pressure in the first embodiment;

FIG. 11 is a diagram illustrating configuration of the scope and the over tube in a second embodiment of the present invention;

FIG. 12 is a diagram illustrating a state close to the scope and the over tube while suctioning fluid in the second embodiment;

FIG. 13 is a diagram illustrating configuration of the scope and the over tube in a third embodiment of the present invention;

FIG. 14 is a diagram illustrating configuration of the scope and the over tube in a fourth embodiment of the present invention;

FIG. 15 is a diagram illustrating configuration of the scope and the over tube in a fifth embodiment of the present invention;

FIG. 16 is a 16-16 sectional view of FIG. 15 illustrating the configuration of the over tube in the fifth embodiment;

FIG. 17 is a 17-17 sectional view of FIG. 15 illustrating the configuration of the over tube in the fifth embodiment;

FIG. 18 is an 18-18 sectional view of FIG. 15 illustrating the configuration of the over tube in the fifth embodiment;

FIG. 19 is a perspective view illustrating configuration of a distal end portion of the over tube constituted as a multi-lumen tube in the fifth embodiment;

FIG. 20 is a diagram illustrating configuration of the scope and the over tube in a sixth embodiment of the present invention;

FIG. 21 is a perspective view illustrating configuration of a small-diameter portion provided at the over tube in the sixth embodiment;

FIG. 22 is a diagram illustrating configuration of the endoscope apparatus in the sixth embodiment; and

FIG. 23 is a diagram illustrating configuration in which a contact sensor is arranged at a balloon in a seventh embodiment of the present invention.

### DETAILED DESCRIPTION OF THE PREFERRED EMBODIMENTS

Embodiments of the present invention will be described below referring to the attached drawings.

### First Embodiment

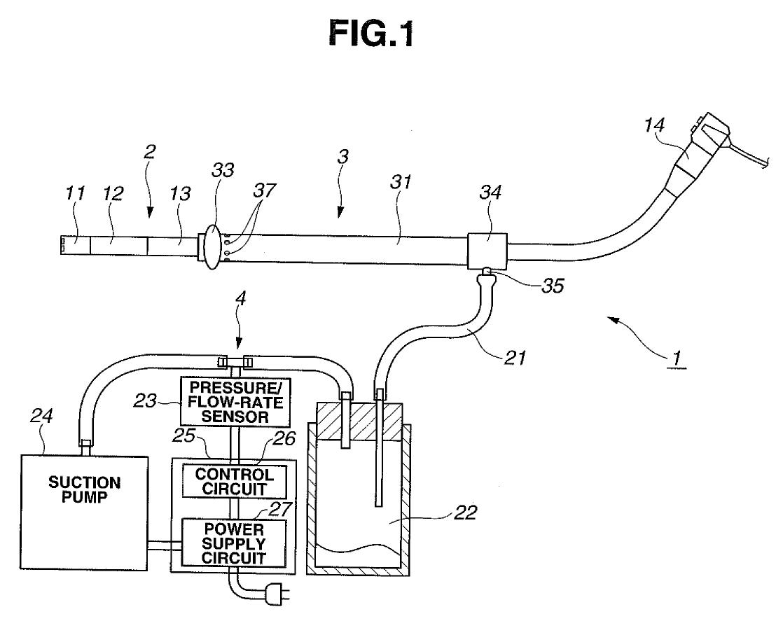

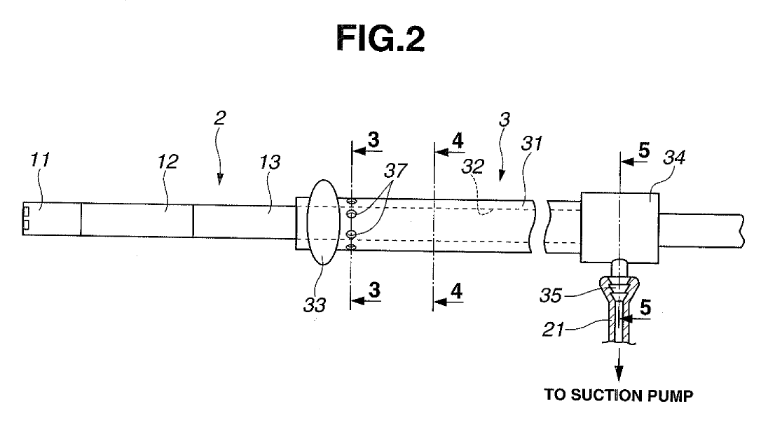

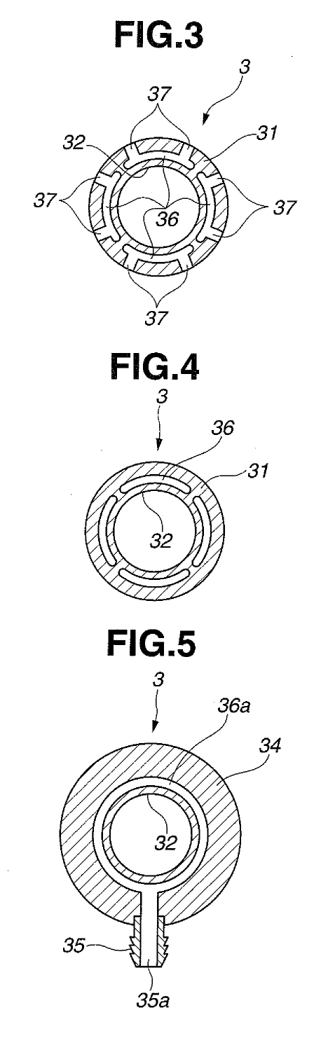

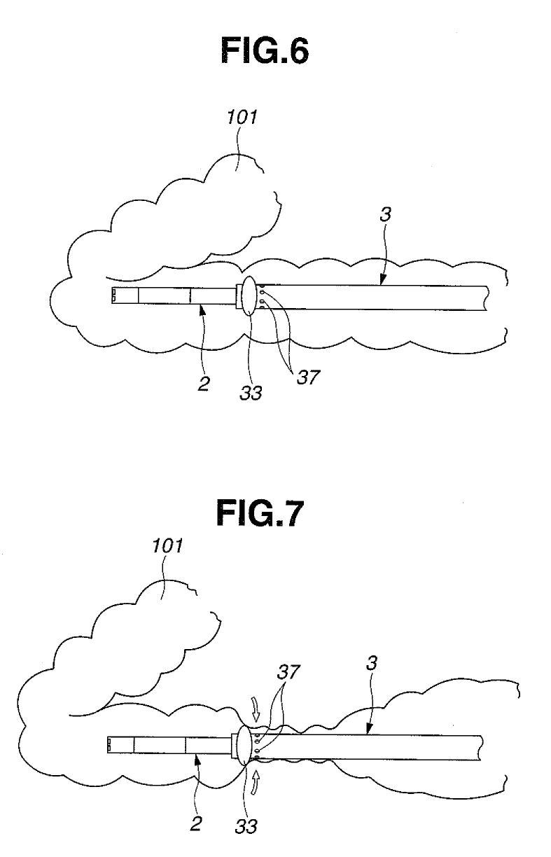

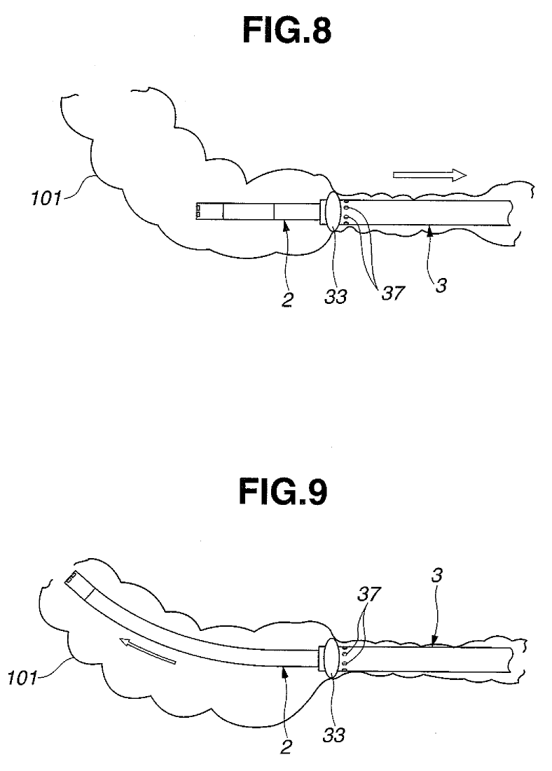

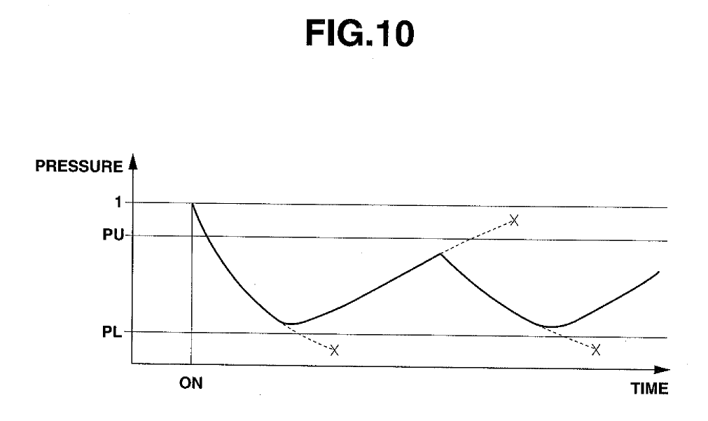

FIGS. 1 to 10 illustrate a first embodiment of the present invention, in which FIG. 1 is a diagram illustrating configuration of an endoscope apparatus, FIG. 2 is a diagram illustrating configuration of a scope and an over tube in more detail, FIG. 3 is a 3-3 sectional view of FIG. 2 illustrating the configuration of the over tube, FIG. 4 is a 4-4 sectional view of FIG. 2 illustrating the configuration of the over tube, FIG. 5 is a 5-5 sectional view of FIG. 2 illustrating the configuration of the over tube, FIG. 6 is a diagram illustrating a state where the scope and the over tube are inserted into a lumen, FIG. 7 is a diagram illustrating a state where the scope and the over tube are inserted into the lumen for suctioning, FIG. 8 is a diagram illustrating a state where the scope and the over tube inserted into the lumen in a suctioning state are pulled so as to make the lumen straight, FIG. 9 is a diagram illustrating a state where the scope is relatively advanced and inserted with respect to the over tube after the lumen is made straight, and FIG. 10 is a line diagram illustrating an example of control so that the pressure of the suctioning becomes an appropriate pressure.

As shown in FIG. 1, an endoscope apparatus 1 comprises a scope 2, which is an endoscope body, an over tube 3, which is an intraluminal insertion portion, and a suctioning device 4.

The scope 2 comprises, from the distal end side to the proximal end side (forward) in the order, a distal-end rigid portion 11 provided with an image pickup optical system, an illuminating optical system and the like, a bending portion 12 for bending the distal-end rigid portion 11 in a desired direction in right and left as well as up and down around an insertion axis, an elongated endoscope insertion portion (hereinafter abbreviated as an insertion portion as appropriate) 13, which is an intraluminal insertion portion comprising the distal-end rigid portion 11 and the bending portion 12, extended further to the proximal end side from the bending portion 12 so as to be inserted into a lumen, and an operation portion 14 connected to the proximal end side of the insertion portion 13 and provided with buttons for bending operation of the bending portion 12, switches for image pickup operations and the like. The operation portion 14 is connected to a video processor and a light source device, not shown, through a universal cord and the like.

The over tube 3 is disposed on an outer peripheral side of the insertion portion 13 of the scope 2 and to be inserted into the lumen of a subject together with the scope 2. The over tube 3 also guides the scope 2, when the scope 2 is relatively advanced/retreated with respect to the over tube 3.

The over tube 3 is provided with an insertion hole 32 (See FIG. 2) for inserting the scope 2 in an inner peripheral side of an over tube body (hereinafter abbreviated as a tube body as appropriate) 31. On the outer periphery of a distal end portion of the over tube 3, a balloon 33, which is a large-diameter portion, is provided. The balloon 33 is in a state expanded in advance to a predetermined size larger than the outer diameter of the tube body 31 and does not expand/contract.

In the vicinity of the proximal end side of the balloon 33, a plurality of (eight in an example shown in FIG. 3) suction opening portions 37 are opened along the circumferential direction of an outer peripheral face of the tube body 31.

These suction opening portions 37 communicate with a suction pipeline 36, which is a fluid suction pipeline provided in the tube body 31 having a predetermined thickness. In an example shown in FIGS. 3 and 4, it is constituted that two suction opening portions 37 communicate with the single suction pipeline 36, that is, four suction pipelines 36 corresponding to eight suction opening portions 37 are provided in the tube body 31 (The over tube 3 is constituted using a multi-lumen tube). These four suction pipelines 36 communicate with each other in an over-tube base end portion 34 provided with a slightly large diameter on the proximal end side of the tube body 31, as shown in FIG. 5, so as to become a single suction pipeline 36a. And the suction pipeline 36a, which is a fluid suction pipeline, communicates with a base hole 35a of a suction base 35 projected on the side of the over-tube base end portion 34.

At the suction base 35, a distal end side of a suction tube 21, which is a fluid suction pipeline, is mounted, and the suction tube 21 is, as shown in FIG. 1, connected to the suctioning device 4 side.

The suctioning device 4 comprises a suction bin 22, a pressure/flow-rate sensor 23, which is a pressure detection portion and flow-rate detection portion, a suction pump 24, and a suction control device 25.

The suction tube 21 from the over tube 3 is connected to the suction pump 24 through the suction bin 22 and the pressure/flow-rate sensor 23. Therefore, the suction pipeline 36 is connected to the suction pump 24.

The suction bin 22 captures mucus, filth and the like suctioned through the suction pipeline 3 6a and the suction tube 21.

The pressure/flow-rate sensor 23 detects a pressure and a flow rate of a fluid flowing through the suction tube 21 such as a gas (or liquid), for example.

The suction pump 24 suctions a fluid in the suction tube 21 and hence, the fluid in the suction pipeline 36.

The suction control device 25 is provided with a control circuit 26 as a control portion and a power supply circuit 27. The power supply circuit 27 supplies power for driving the suction pump 24 and is controlled by the control circuit 26.

The control circuit 26 is connected to the pressure/flow-rate sensor 23 and controls the suction pump 24 through the power supply circuit 27 based on the pressure or flow rate of the fluid in the suction tube 21 detected by the pressure/flow-rate sensor 23 so that the pressure of the fluid in the lumen in the vicinity of the suction opening portion 37 becomes an appropriate pressure. That is, since a pipe diameter and a pipeline length of the suction tube 21 as well as a pipe diameter and a pipeline length of the suction pipeline 36 are set in advance, if the pressure or flow rate of the fluid is detected by the pressure/flow-rate sensor 23 provided at the suction tube 21, it is possible to estimate a degree of the pressure of the fluid in the lumen in the vicinity of the suction opening portion 37 according to the pressure or flow rate. Therefore, the control circuit 26 controls suctioning by the suction-pump 24 so as to keep the pressure in the lumen appropriate based on a relation between the pressure or flow rate detected by the pressure/flow-rate sensor 23 and the pressure in the lumen in the vicinity of the suction opening portion 37. The control of the pressure by the control circuit 26 will be described later referring to FIG. 10.

Next, referring to FIGS. 6 to 9, an action of insertion into the lumen by the endoscope apparatus 1 will be described. Here, an intestine 101 is used as an example of a lumen.

First, as shown in FIG. 6, the scope 2 and the over tube 3 are inserted into the intestine 101. This insertion is conducted through an anus, for example. Since the intestine 101 is provided with a bending portion as shown in FIG. 6 and smooth insertion might be difficult only by pushing in, the insertion is stopped temporarily when the insertion has reached before the bending portion.

Next, as shown in FIG. 7, by operating the suction pump 24, air, for example, in the intestine 101 is suctioned from the suction opening portion 37. By the operation, the pressure inside the intestine 101 in the vicinity of the suction opening portion 37 becomes more negative than the periphery, and the intestine 101 is drawn close to the over tube 3.

In this state, by pulling the over tube 3 and the scope 2 integrally toward the hand side, since the balloon 33 as the large-diameter portion gets hooked and gripped by the intestine 101, the intestine 101 is pulled toward the hand side with movement of the over tube 3 and the scope 2 and made straight as shown in FIG. 8 (Particularly, the bending portion of the intestine 101 is made straight).

In this state, by inserting the scope 2 so that the scope is relatively advanced with respect to the over tube as shown in FIG. 9, smooth insertion is possible since the intestine 101 has been made straight.

Then, referring to FIG. 10, control of a suctioning pressure by the control circuit 26 will be described.

The control circuit 26 stores an upper-limit pressure value PU and a lower-limit pressure value PL in advance so that the pressure in the vicinity of the suction opening portion 37 falls within an appropriate range. Here, the upper-limit pressure value PU is lower than 1 barometric pressure, which is the atmospheric pressure, and it is a pressure required when the intestine 101 is drawn toward the over tube 3 side (that is, the upper-limit pressure value PU is a pressure such that an action to draw the intestine 101 toward the over tube 3 side is considered to become insufficient if the pressure is higher than the value). Also, the lower-limit pressure value PL is a pressure such that a mucus membrane or the like of the intestine 101 is not suctioned further into the suction opening portion 37 (conversely, if the pressure becomes lower than the lower-limit pressure value PL, it is determined that there is a possibility of attachment of the mucus membrane or the like of the intestine 101 into the suction opening portion 37).

When the suction pump 24 is turned on, the pressure detected by the pressure/flow-rate sensor 23 is gradually lowered as shown in FIG. 10.

At this time, if the control circuit 26 determines that the pressure would become lower than the lower-limit pressure value PL with the pressure drop speed, the circuit slows the suction speed of the suction pump 24 or stops suction by so-called PID control so as to restrict further pressure drop.

On the other hand, if the control circuit 26 determines that the pressure would become higher than the upper-limit pressure value PU with the pressure rise speed, the circuit resumes the suctioning by the suction pump 24 or accelerates the suction speed so as to restrict further pressure rise.

As mentioned above, the control circuit 26 executes control so that the pressure in the lumen in the vicinity of the suction opening portion 37 is not more than the upper-limit pressure value PU and not less than the lower-limit pressure value PL. Here, an example of control so that the pressure in the lumen becomes not more than the upper-limit pressure value PU and not less than the lower-limit pressure value PL is shown, but it is needless to say that a target pressure value is set and pressure is controlled so that the target pressure value is maintained. Alternatively, the upper-limit pressure value PU and the lower-limit pressure value PL may be set at a value proximate to the target pressure value. It is needless to say that the upper-limit pressure value PU, the lower-limit pressure value PL or the target pressure value may be configured so that an operator can set as desired.

According to the above first embodiment, instead of conventional configuration of a type in which a balloon is expanded according to an inner diameter of a lumen, since the embodiment is configured that the lumen is suctioned so as to be drawn to the balloon side and the suction pressure is controlled to fall within an appropriate range, the balloon can surely grip the lumen without lack or excess regardless of the inner diameter of the lumen. Therefore, work to make the bending lumen straight can be more surely executed regardless of the inner diameter of the lumen.

Also, since a plurality of suction opening portions are provided so that even if some of the suction opening portions are blocked, suctioning can be continued from the other suction opening portions, enabling sure suctioning.

In the case of configuration that only a single suction opening portion is provided for a single suction pipeline, there is a possibility that if a suction opening portion is blocked, the pressure rapidly drops and the lumen is drawn in from the suction opening portion, but in the present embodiment, since such configuration is employed that the plurality of suction opening portions communicates finally with a single suction pipeline, even if some suction opening portions are blocked, fluid is suctioned from the other suction opening portions and rapid pressure drop can be prevented.

In addition, even if all the suction opening portions are blocked, since the control circuit 26 executes pressure control so as to stop the suctioning or to slow the suction speed, the rapid pressure drop would not occur.

As mentioned above, membrane's being stuck to the suction opening portion can be prevented and damage on the mucus membrane can be prevented, too.

Moreover, with the configuration of the present embodiment, since work to expand, contract or the like of the balloon is not required, a treatment time can be reduced and operations are simplified.

### Second Embodiment

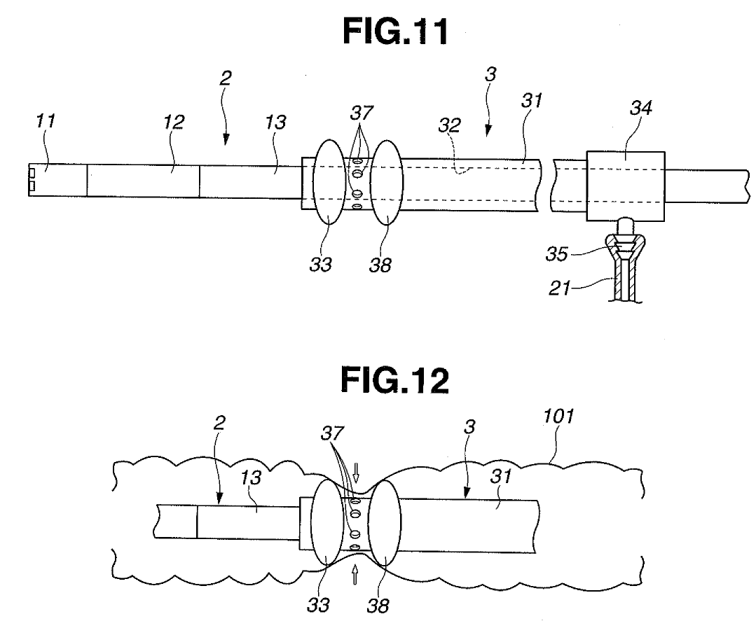

FIGS. 11 and 12 show a second embodiment of the present invention, in which FIG. 11 is a diagram illustrating configuration of the scope and the over tube, and FIG. 12 is a diagram illustrating a state in the vicinity of the scope and the over tube when a fluid is being suctioned. In the second embodiment, the same reference numerals are given to the portions similar to those in the first embodiment and the description will be omitted, and only the differences will be mainly described.

The over tube 3 in the present embodiment has, as shown in FIG. 11, a second balloon 3 8 as a large-diameter portion is further provided at the proximal end side of the suction opening portion 37 in addition to the configuration of the first embodiment.

With such configuration, if a fluid is suctioned from the suction opening portion 37, when the intestine 101 as a lumen is drawn the suctioned fluid is mainly a fluid in the intestine 101 between the first balloon 33 and the second balloon 38 (See FIG. 12). That is, with the configuration of the first embodiment, since it is necessary to suction the fluid in the intestine 101 on the proximal end side from the balloon 33, suctioning should be continued even after the intestine 101 has been drawn to the over tube 3 side. On the other hand, according to the present embodiment, after the intestine 101 has been drawn toward the over tube 3 side once, it is only necessary to keep on applying a suction pressure or to suction only some fluid leaking to between the first balloon 33 and the second balloon 38.

According to the second embodiment, substantially similar advantages to the above first embodiment are exerted and more efficient suctioning is enabled at the same time.

### Third Embodiment

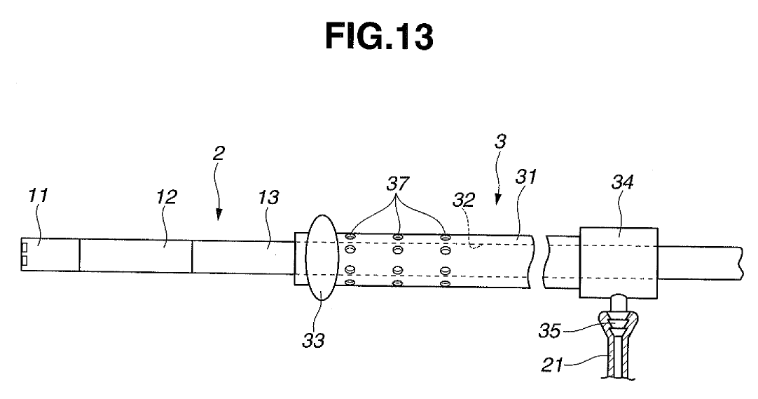

FIG. 13 shows a third embodiment of the present invention and is a diagram illustrating configuration of the scope and the over tube. In the third embodiment, the same reference numerals are given to the portions similar to those in the first and second embodiments and the description will be omitted, and only the differences will be mainly described.

In the present embodiment, the suction opening portion 37 is provided in plural at different positions in a direction of an insertion axis. That is, the over tube 3 of the present embodiment is configured substantially similarly to that of the first embodiment, but the plurality of suction opening portions 37 disposed in the circumferential direction are disposed in plural pairs at different positions in the insertion-axis direction.

Action of the third embodiment is substantially similar to that of the first embodiment.

According to such third embodiment, substantially similar advantages to those of the above first embodiment are exerted and at the same time, since the number of suction opening portions 37 is increased, an opening area is enlarged, by which time required for suctioning of fluid can be reduced and inspection time can be also reduced. Moreover, since the number of suction opening portions 37 is increased and there are the suction opening portions 37 at different positions in the insertion-axis direction, a possibility that all the suction opening portions 37 are blocked can be further reduced.

### Fourth Embodiment

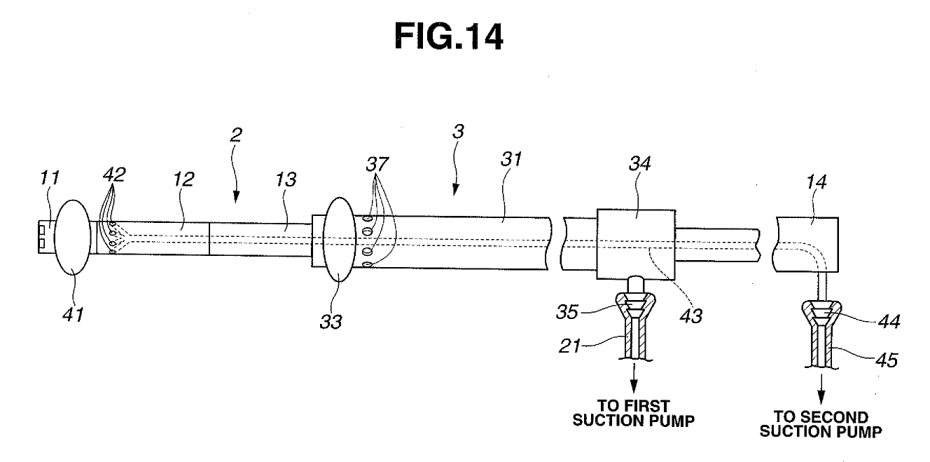

FIG. 14 shows a fourth embodiment of the present invention and is a diagram illustrating configuration of the scope and the over tube. In the fourth embodiment, the same reference numerals are given to the portions similar to those in the first to third embodiments and the description will be omitted, and only the differences will be mainly described.

In the above first embodiment, the balloon 33 and the suction opening portion 37 are provided at the over tube 3, but in the present embodiment, in addition to the above, a balloon 41 as a large-diameter portion and a suction opening portion 42 are further provided also at the scope 2.

That is, at the distal-end rigid portion 11 of the scope 2, the balloon 41 as the large-diameter portion is provided. And in the vicinity of the balloon 41 on the proximal end side, a plurality of the suction opening portions 42 are opened along the circumferential direction of the outer peripheral face of the distal-end rigid portion 11. These plural suction opening portions 42 communicate with a suction pipeline 43 as a fluid suction pipeline disposed in the scope 2.

The suction pipeline 43 is connected to a suction tube 45 as a fluid suction pipeline through a suction base 44 disposed on the side of the operation portion 14, for example, after being disposed to the operation portion 14 on the hand side. This suction tube 45 is connected to a second suction pump, not shown, that is, by operating the second suction pump, suctioning is carried out from the suction opening portion 42.

In the endoscope apparatus 1 with such configuration, the action that the balloons 33, 41 can surely grip the lumen without lack or excess by suctioning the fluid in the lumen so as to draw in the lumen is similar to the first embodiment.

Also, in the endoscope apparatus 1 of the present embodiment, the insertion procedure into the lumen is slightly different from that in the first embodiment, that is, the procedure is based on a so-called double-balloon type endoscope apparatus.

That is, first, the scope 2 and the over tube 3 are inserted into the intestine 101 (See FIG. 6).

Next, the suction pump 24 is operated so as to draw in the intestine 101 toward the over tube 3 (See FIG. 7).

In this state, by pulling the over tube 3 and the scope 2 integrally toward the hand side, the intestine 101 is drawn in forward with the movement of the over tube 3 and the scope 2 and made straight (See FIG. 8).

In this state, the scope 2 is inserted so as to advance relatively with respect to the over tube 3 (See FIG. 9).

Next, by carrying out suctioning from the suction opening portion 42 of the scope 2, the intestine 101 is drawn in to the balloon 41 of the scope 2 and then, suctioning from the suction opening portion 37 of the over tube 3 is stopped.

As a result, the intestine 101 is brought into a state not drawn in to the over tube 3, and in this state, the over tube 3 is inserted along the scope 2.

After that, suctioning is carried out from the suction opening portion 37 so as to keep drawing the intestine 101 to the over tube 3, and then, suctioning from the suction opening portion 42 of the scope 2 is stopped.

After that, by repeating an operation substantially similar to the above, that is, though described in partial duplicate, a procedure of:

Suction of the over tube 3
↓

Suction stop of the scope 2
↓

Pulling of the over tube 3 and the scope 2 toward the hand side
↓

Insertion of only the scope 2
↓

Suction of the scope 2
↓

Suction stop of the over tube 3
↓

Insertion of only the over tube 3,

the scope 2 and the over tube 3 can be inserted into a deeper portion in the lumen.

When the over tube 3 and the scope 2 are pulled toward the hand side, in order to enhance the gripping force, the pulling is preferably carried out while suctioning both by the over tube 3 and the scope 2 is conducted. At this time, a procedure of

Suction of the over tube 3
↓

Pulling of the over tube 3 and the scope 2 toward the hand side
↓

Suction stop of the scope 2
↓

Insertion of only the scope 2
↓

Suction of the scope 2
↓Suction stop of the over tube 3
↓

Insertion of only the over tube 3

is repeated.

According to such fourth embodiment, an effect substantially similar to that of the above first to third embodiments is exerted, and since the lumen can also be gripped by the balloon 41 provided at the scope 2 by carrying out suctioning, when the over tube 3 is inserted along the scope 2, the distal end position of the scope 2 is hard to be displaced, by which smooth and efficient insertion of the over tube 3 without insertion waste is enabled.

### Fifth Embodiment

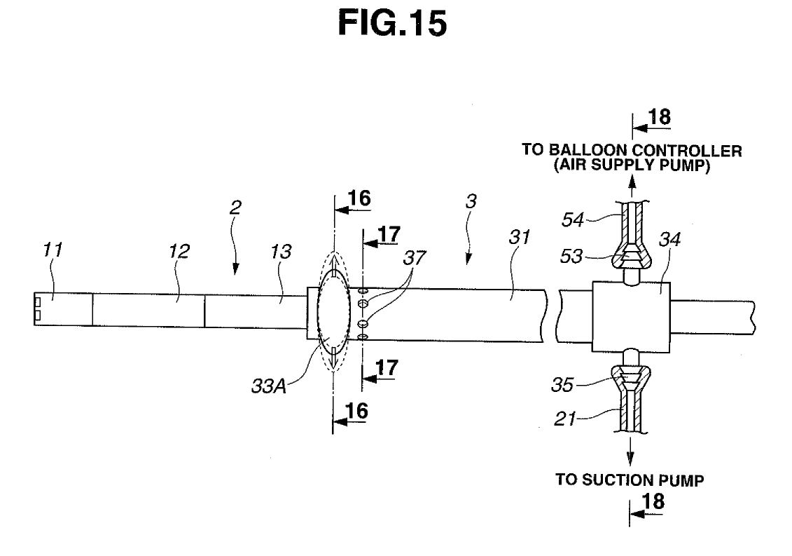

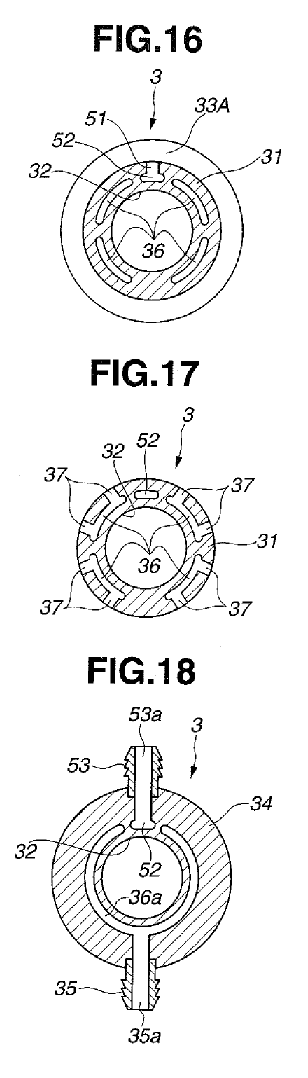

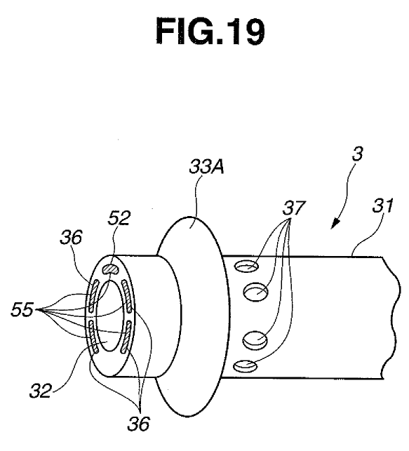

FIGS. 15 to 19 show a fifth embodiment of the present invention, in which FIG. 15 is a diagram illustrating configuration of the scope and the over tube, FIG. 16 is a 16-16 sectional view of FIG. 15 illustrating the configuration of the over tube, FIG. 17 is a 17-17 sectional view of FIG. 15 illustrating the configuration of the over tube, FIG. 18 is an 18-18 sectional view of FIG. 15 illustrating the configuration of the over tube, and FIG. 19 is a perspective view illustrating configuration of a distal end portion of the over tube constituted as a multi-lumen tube.

In the fifth embodiment, the same reference numerals are given to the portions similar to those in the first to fourth embodiments and the description will be omitted, and only the differences will be mainly described.

A balloon 33A as a large-diameter portion of the present embodiment is configured capable of expansion/contraction, unlike the first embodiment.

That is, at the tube body 31 in the balloon 33A, an opening portion 51 for balloon is opened. This balloon opening portion 51 communicates with a balloon pipeline 52 provided as a pipeline independent of the above suction pipeline 36 in the tube body 31 with a predetermined thickness.

The balloon pipeline 52 is, as shown in FIGS. 16 to 18, disposed in the insertion-axis direction in the tube body 31 and communicates with a base hole 53a of a base 53 for balloon projected on the side of the over-tube base end portion 34.

At the base 53 for balloon, a distal end side of a tube 54 for balloon is mounted, and the tube 54 for balloon is connected to a balloon controller (including an air supply pump and the like), not shown.

By this arrangement, the expansion/contraction of the balloon 33A can be controlled through the balloon controller.

The over tube 3 of the present embodiment is also configured using a multi-lumen tube, but as shown in FIG. 19, an opening on the distal end side is blocked by pouring a silicon rubber 55 and the like into the distal end side. A manufacturing method of the over tube 3 using such multi-lumen tube is not limited to the present embodiment but can be similarly applied to the other embodiments. Also, the proximal end side of the multi-lumen tube is fitted in the multi-lumen tube or blocked by the over-tube base end portion 34 configured as another member, for example.

Moreover, it is needless to say that the configuration of the balloon capable of expansion/contraction as shown in the fifth embodiment may be applied to the other embodiments.

According to such fifth embodiment, an effect substantially similar to that of the above first embodiment is exerted and since a diameter of the balloon can be adjusted, application in a wider range for various lumens with different inner diameters becomes possible.

### Sixth Embodiment

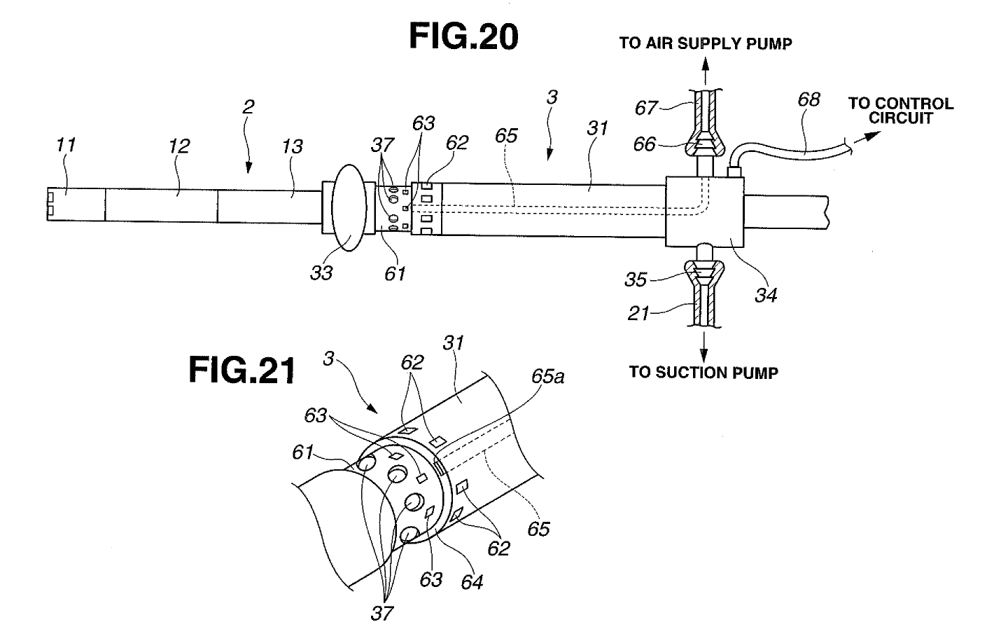

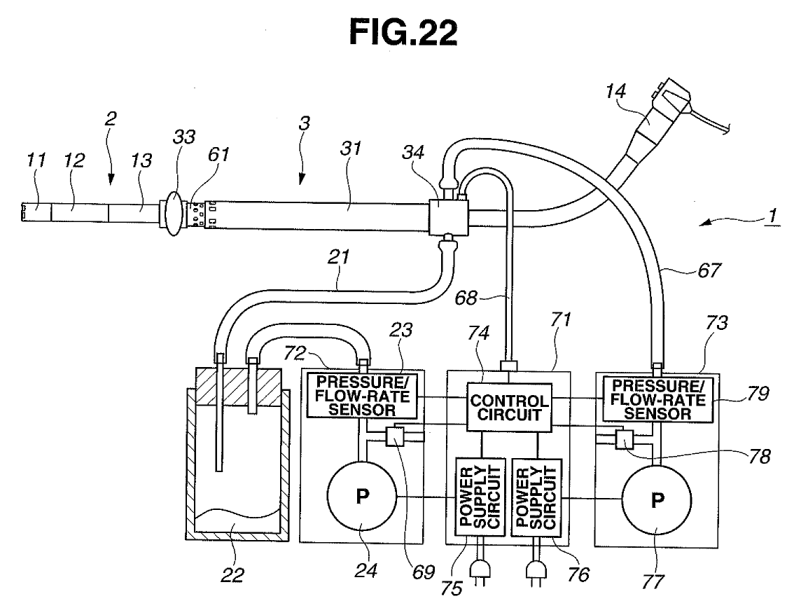

FIGS. 20 to 22 show a sixth embodiment of the present invention, in which FIG. 20 is a diagram illustrating configuration of the scope and the over tube, FIG. 21 is a perspective view illustrating configuration of a small-diameter portion provided at the over tube, and FIG. 22 is a diagram illustrating configuration of the endoscope apparatus. In the sixth embodiment, the same reference numerals are given to the portions similar to those in the above first to fifth embodiments and the description will be omitted, and only the differences will be mainly described.

The endoscope apparatus 1 of the present embodiment is provided with, in addition to the configuration of the endoscope apparatus 1 of the above first embodiment, an air supply function for avoiding excessive suctioning and also a contact sensor for detecting a suction state.

That is, the tube body 31 of the present embodiment is provided with, in the vicinity of the proximal end side of the balloon 33, as shown in FIGS. 20 and 21, a small-diameter portion 61. And at the small-diameter portion 61, a plurality of suction opening portions 37 are opened along the circumferential direction and a plurality of second contact sensors 63 as contact detection portions are disposed along the circumferential direction.

At a normal diameter portion of the tube body 31 adjacent to the small-diameter portion 61, a plurality of first contact sensors 62 as the contact detection portions are disposed along the circumferential direction.

The first contact sensors 62 and the second contact sensors 63 detect whether or not contact is made with a luminal portion or the like, and a positional relation in the radial direction with the balloon 33 and the suction opening portion 37 around the insertion axis is as follows:

Outer diameter of the balloon 33>Diameter of the first contact sensor 62 at an installed position>Diameter of the second contact sensor 63 at an installed position≧Diameter of the suction opening portion 37 And a signal line, not shown, is connected to the first contact sensors 62 and the second contact sensors 63, and the signal line is disposed through the tube body 31 and further, after the line is disposed in a signal cable 68 extended from the over-tube base end portion 34, the line is connected to a control circuit 74 as a control portion of a suction control device 71 shown in FIG. 22.

On the other hand, at a stepped portion 64 connecting the above small-diameter portion 61 and the normal diameter portion of the tube body 31, an air-supply opening portion 65a is opened, and the air-supply opening portion 65a communicates with an air-supply pipeline 65 provided as a pipeline independent of the above suction pipeline 36 in the tube body 31 having a predetermined thickness.

The air-supply pipeline 65 is, as shown in FIG. 20, disposed in the insertion-axis direction in the tube body 31 and then, communicates with an air-supply base 66 projected on the side of the over-tube base end portion 34.

On the air-supply base 66, a distal end side of the air-supply tube 67 is mounted, and the air-supply tube 67 is connected to the air-supply pump 77 shown in FIG. 22.

And the suctioning device 4 of the present embodiment is provided with the suction bin 22, a suction pump device 72, a suction control device 71, and an air-supply pump device 73.

The suction bin 22 is similar to that in the above first embodiment.

The suction pump device 72 is provided with the suction pump 24, the pressure/flow-rate sensor 23, and an emergency open valve 69. Here, the emergency open valve 69 discharges the pressure of the suction tube 21 and the suction pipeline 36 into the air by having the suction pressure by the suction pump 24 communicate with the air. And the emergency open valve 69 and the pressure/flow-rate sensor 23 are connected to a control circuit 74 in the suction control device 71 so as to be controlled.

The air-supply pump device 73 is provided with an air-supply pump 77, a pressure/flow-rate sensor 79, and an emergency open valve 78. Here, the air-supply pump 77 feeds air to the air-supply tube 67. The pressure/flow-rate sensor 79 detects a pressure and a flow rate of a gas fed from the air-supply pump 77. The emergency open valve 78 discharges the pressure of the air-supply tube 67 into the air by having air supply by the air-supply pump 77 escape into the air side. And the emergency open valve 78 and the pressure/flow-rate sensor 79 are connected to the control circuit 74 in the suction control device 71 so as to be controlled.

The suction control device 71 is provided with the control circuit 74, a power supply circuit 75, and a power supply circuit 76. The power supply circuit 75 supplies power for driving the suction pump 24 and is controlled by the control circuit 74. The power supply circuit 76 supplies power for driving the air-supply pump 77 and is controlled by the control circuit 74. And the control circuit 74 determines a contact state of the lumen with the over tube 3 in the vicinity of the balloon 33, the pressure and flow rate of the fluid in the suction tube 21, and the pressure and flow rate of the gas in the air-supply tube 67, based on sensor outputs from the above first contact sensors 62 and the second contact sensors 63 and sensor outputs from the pressure/flow-rate sensor 23 and the pressure/flow-rate sensor 79, controls the suction pump 24 through the power supply circuit 75 or controls the air-supply pump 77 through the power supply circuit 76 as necessary so that the pressure of the fluid in the lumen in the vicinity of the suction opening portion 37 becomes an appropriate pressure.

Specifically, the control by the control circuit 74 is carried out as follows, for example.

First, if the majority of the first contact sensors 62 provided in plural senses a contact state, the control circuit 74 controls the suction pump 24 through the power supply circuit 75 so as to weaken the suction pressure (that is, the suction pressure is somewhat brought close to the atmospheric pressure).

Also, if the majority of the second contact sensors 63 provided in plural senses the contact state, the control circuit 74 controls the suction pump 24 through the power supply circuit 75 so as to stop the suctioning.

Moreover, if all the first contact sensors 62 and the second contact sensors 63 sense the contact state, the control circuit 74 determines the state as excessive suctioning and carries out air supply by the air-supply pump 77 through the power supply circuit 76. By this operation, air is supplied into the lumen through the air-supply pipeline 65. After that, when the total contact state of the first contact sensors 62 and the second contact sensors 63 is avoided (that is, at least one of the contact sensors 62, 63 senses a non-contact state), control is executed so that the air supply by the air-supply pump 77 is automatically stopped. Here, the air supply by the air-supply pump 77 is automatically stopped when at least one contact sensor 62, 63 senses the non-contact state, but instead of that, the air supply by the air-supply pump 77 may be automatically stopped when all the contact sensors 62, 63 sense the non-contact state.

And, the control circuit 74 discharges the suction pressure to the air by opening the emergency open valve 69 if the suction pressure detected by the pressure/flow-rate sensor 23 becomes lower than a value set in advance or if a pressure drop rate exceeds a rate set in advance.

Similarly, the control circuit 74 discharges the air-supply pressure to the air by opening the emergency open valve 78 if the air-supply pressure detected by the pressure/flow-rate sensor 79 exceeds a value set in advance or if a pressure rise rate exceeds a rate set in advance.

Here, the control circuit 74 controls the emergency open valves 69, 78 mainly based on the suction pressure or the air-supply pressure, but instead of this or in addition to this, the emergency open valves 69, 78 may be controlled based on a suction flow rate or air-supply flow rate.

According to such sixth embodiment, an effect substantially similar to that of the above first embodiment is exerted and since the plurality of contact sensors 62, 63 are arranged in the plurality of positions different in the radial direction around the insertion axis so as to detect the contact state between the over tube 3 and the lumen, more appropriate and detailed control of the suction pressure is enabled.

Moreover, by providing the air-supply opening portion 65a in the vicinity of the suction opening portion 37 and supplying air if excessive suctioning is detected so as to positively prevent being stuck of the intestine mucus membrane or the like, damage on the mucus membrane or the like can be prevented more assuredly.

And since the emergency open valve 69 is connected to the suction pump 24 and the emergency open valve 78 is connected to the air-supply pump 77, if the suctioning or air supply exceeds a predetermined range, the pressure can be discharged to the air immediately, by which the suctioning or air supply can be returned to the predetermined range in an urgent manner.

In the above, the first contact sensors 62 and the second contact sensors 63 are described as sensors of a type to detect if there has been contact or not but not limited to that, they may be pressure sensors (contact-type pressure sensors) of a type to detect if there has been contact or not and moreover, if there has been contact, to detect its contact pressure, for example.

If such configuration is employed, by averaging values detected from the plurality of first contact sensors 62, if the averaged value becomes larger than a predetermined set value, the control circuit 74 controls the suction pump 24 through the power supply circuit 75 so as to weaken the suction pressure (that is, the suction pressure is brought somewhat close to the atmospheric pressure).

Moreover, by averaging the values detected from the plurality of second contact sensors 63, if the averaged value becomes larger than the predetermined set value, the control circuit 74 controls the suction pump 24 through the power supply circuit 75 so as to stop the suctioning.

If such configuration is employed, it becomes possible to distinguish whether the lumen is accidentally brought into contact with the contact sensor or the lumen contacts the contact sensor with a pressure, and more accurate and detailed suction pressure control is enabled, which is an advantage.

### Seventh Embodiment

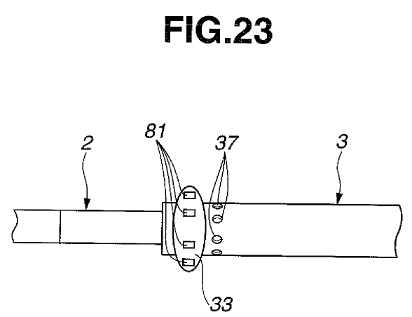

FIG. 23 shows a seventh embodiment of the present invention and it is a diagram illustrating configuration in which a contact sensor is arranged at a balloon. In the seventh embodiment, the same reference numerals are given to the portions similar to those in the first to sixth embodiments and the description will be omitted, and only the differences will be mainly described.

At a portion with the largest diameter on the outer surface of the balloon 33 in the present embodiment, a plurality of contact sensors 81 as contact detection portions are disposed along the circumferential direction. The contact sensor 81 may be either a type to detect only presence of contact or a type to detect presence of the contact and a pressure at contact.

Since the contact sensor 81 provided on the outer surface of the balloon 33 detects a grip state between the balloon 33 and the lumen, if contact is detected by all the contact sensors 81 or if an average value of the contact pressure of all the contact sensors 81 is larger than a predetermined value, it is determined that the lumen can be gripped by the balloon 33.

Conversely, if contact by all the contact sensors 81 is not detected or the average value of the contact pressure of all the contact sensors 81 is at the predetermined value or less, the grip by the balloon 33 may be made assured by suctioning by the suction pump 24.

The configuration of the seventh embodiment may be provided instead of the configuration of the above sixth embodiment or may be provided in addition to the configuration of the sixth embodiment.

An example in which the contact sensor 81 is provided on the surface of the balloon 33 was described above, but the sensor may be provided inside the balloon 33. At this time, a type of sensor capable of detecting a pressure is disposed so as to detect if the gripping force on the lumen by the balloon 33 is sufficient or not.

Besides, if the balloon 33 is a type capable of expansion/contraction, instead of provision on the surface or inside the balloon 33, the pressure sensor may be provided at a balloon control unit for expanding/contracting the balloon 33.

According to such seventh embodiment, an effect substantially similar to that of the above first embodiment is exerted and whether the grip of the lumen by the balloon 33 can be surely executed or not can be detected.

A balloon is cited as an example as the large-diameter portion in the above, but the portion is not limited to the balloon as long as the portion is configured with a diameter larger than the normal portion of the intraluminal insertion portion and in a shape capable of gripping the lumen.

The endoscope apparatus in each of the above embodiments is not limited to medical use but the apparatus may naturally be used for industry, too.

Having described the preferred embodiments of the invention referring to the accompanying drawings, it should be understood that the present invention is not limited to those precise embodiments and various changes and modifications there could be made by one skilled in the art without departing from the spirit or scope of the invention as defined in the appended claims.

What is claimed is:

1. An endoscope apparatus comprising:
an intraluminal insertion portion to be inserted into a lumen;
a large-diameter portion provided at the intraluminal insertion portion;
a fluid suction pipeline at least provided at the intraluminal insertion portion so that a suction opening portion is opened on an outer surface of the intraluminal insertion portion at a proximal end side from the large-diameter portion;
a suctioning device to which a proximal end side of the fluid suction pipeline is connected for suctioning a fluid in the fluid suction pipeline;
a pressure detection portion for detecting a pressure of the fluid at least at a single position from inside the lumen to which the intraluminal insertion portion is inserted to the suctioning device through the fluid suction pipeline; and
a control portion for controlling the suctioning device so that the pressure of the fluid in the lumen in the vicinity of the suction opening portion becomes an appropriate pressure based on the pressure detected by the pressure detection portion.

2. The endoscope apparatus according to claim 1, wherein
the control portion executes control so that the pressure of the fluid in the lumen in the vicinity of the suction opening portion becomes an appropriate pressure by controlling the suctioning device so that the pressure detected by the pressure detection portion does not become lower than a predetermined lower limit value.

3. The endoscope apparatus according to claim 2, wherein
the control portion executes control so that the pressure of the fluid in the lumen in the vicinity of the suction opening portion becomes an appropriate pressure by further controlling the suctioning device so that the pressure detected by the pressure detection portion does not become higher than a predetermined upper limit value that is higher than the predetermined lower limit value.

4. The endoscope apparatus according to claim 1, wherein
the pressure detection portion detects the pressure of the fluid in the fluid suction pipeline; and
the control portion estimates the pressure of the fluid in the lumen in the vicinity of the suction opening portion and executes control so that the pressure becomes an appropriate pressure based on the pressure detected by the pressure detection portion.

5. The endoscope apparatus according to claim 1, further comprising
a contact detection portion for detecting contact between the lumen and at least one of the large-diameter portion and the intraluminal insertion portion in the vicinity of the large-diameter portion, and wherein
the control portion executes control so that the pressure of the fluid in the lumen in the vicinity of the suction opening portion becomes an appropriate pressure further based on whether the contact with the lumen is detected by the contact detection portion or not.

6. The endoscope apparatus according to claim 5, wherein
the contact detection portion farther detects a pressure at contact; and
the control portion executes control so that the pressure of the fluid in the lumen in the vicinity of the suction opening portion becomes an appropriate pressure further based on the pressure at contact detected by the contact detection portion.

7. The endoscope apparatus according to claim 1, further comprising
a flow-rate detection portion for detecting a flow rate of the fluid flowing through the fluid suction pipeline, and wherein
the control portion executes control so that the pressure of the fluid in the lumen in the vicinity of the suction opening portion becomes an appropriate pressure further based on the flow rate detected by the flow-rate detection portion.

8. The endoscope apparatus according to claim 1, wherein
the suction opening portions are provided on the outer surface of the intraluminal insertion portion so as to be opened in plural; and
at least two of the plural suction opening portions are configured to communicate with a single common pipeline.

9. The endoscope apparatus according to claim 1, wherein
the intraluminal insertion portion includes an endoscope insertion portion.

10. The endoscope apparatus according to claim 1, wherein
the intraluminal insertion portion includes an over tube disposed on an outer peripheral side of an endoscope insertion portion.
